SEQUENCE LISTING (3 sequences)

SEQ ID NO: 1 - protein, 16 aa, Artificial Sequence
  matrix metalloprotease degradable crosslinker
GCREGPQGIWGQERCG

SEQ ID NO: 2 - protein, 16 aa, Artificial Sequence
  matrix metalloprotease non-degradable crosslinker
GCREGDQGIAGFERCG

SEQ ID NO: 3 - protein, 11 aa, Artificial Sequence
  Cell adhesive peptide
GCGYGRGDSPG

(19) United States
(12) Patent Application Publication
(10) Pub. No.: US 2021/0220388 A1
(43) Pub. Date: Jul. 22, 2021

(54) THERAPEUTIC HYDROGEL MATERIAL AND METHODS OF USING THE SAME

(71) Applicant: THE REGENTS OF THE UNIVERSITY OF CALIFORNIA, Oakland, CA (US)

(72) Inventors: Tatiana Segura, Durham, NC (US); Stanley Thomas Carmichael, Sherman Oaks, CA (US); Lina R. Nih, Redondo Beach, CA (US)

(73) Assignee: THE REGENTS OF THE UNIVERSITY OF CALIFORNIA, Oakland, CA (US)

(21) Appl. No.: 17/054,421

(22) PCT Filed: May 10, 2018
(86) PCT No.: PCT/US2019/031774

§ 371 Date: Nov. 10, 2020

Related U.S. Application Data
(60) Provisional application No. 62/669,862, filed on May 10, 2018.

(51) Int. Cl. A61K 31/727; A61K 9/14; A61K 9/06; A61K 47/36; A61K 47/18; A61K 47/42; A61P 9/10

(52) U.S. Cl. CPC A61K 31/727; A61K 9/14; A61K 9/06; A61B 17/1695; A61K 47/18; A61K 47/42; A61P 9/10; A61K 47/36

(57) ABSTRACT

A therapeutic hydrogel material includes a hyaluronic acid-based hydrogel matrix containing naked heparin nanoparticles distributed and entrained within the matrix. The naked heparin nanoparticles contained in the matrix are not immobilized to any other molecules at the time of delivery. In one aspect of the invention, the therapeutic hydrogel material is used to repair ischemic tissue in a subject (e.g., mammal). The therapeutic hydrogel material may also be used to treat wounds or other damaged tissue. To treat the subject or patient, the site of application is located and the therapeutic hydrogel material is injected or otherwise delivered (with or without a delivery device) to the delivery location along with a crosslinker.

### RELATED APPLICATION

This application claims priority to U.S. Provisional Patent Application No. 62/669,862 filed on May 10, 2018, which is hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with Government support under Grant No. NS079691, awarded by the National Institutes of Health. The Government has certain rights in the invention.

### TECHNICAL FIELD

The technical field generally relates to therapeutic materials that are used to reduce inflammation and repair tissues. In particular, the field of the invention relates to a hydrogel-based therapeutic material that incorporates bare heparin nanoparticles therein. That is to say, the heparin nanoparticles that are entrained within the hydrogel are not bound to any other growth factor or the like.

### BACKGROUND

Nanoparticles have been widely studied and have been investigated for potential applications in biomedical, optical, and electronic fields. Nanoparticles have drawn interest based on properties they exhibit such as, for example, their surface to mass ratio and the reactivity of their surfaces. Nanoparticles may be formed in a number of shapes and types. These include tubes, rods, spheres, and the like.

Heparin is a well-known naturally occurring anticoagulant and antithrombotic in medicinal applications. Heparin that is used in hospital settings is also referred to as unfractionated heparin (UFH), which is used as an anticoagulant (blood thinner). Heparin as been used in nanoparticle synthesis. For example, heparin-based nanoparticles have been made form gold and silver, metal oxides, silica and chitosan, poly(lactide-co-glycolide). Nanoparticles based on heparin have been used for cancer treatment, imaging, and detection. See, e.g., Rodriguez-Torres et al., Heparin-Based Nanoparticles: An Overview of Their Applications, Journal of Nanomaterials, Article ID 9780489, 8 pages, 2018. Heparin nanoparticles have also been used a therapeutic material where Vascular Endothelial Growth Factor (VEGF) is immobilized to the heparin nanoparticles. See International Patent Publication No. WO 2018/187184. There is a need for additional applications and uses of heparin nanoparticles and in particular heparin nanoparticles that do not exhibit the conventional blood thinning properties of heparin yet retain the ability to sequester cytokines.

### SUMMARY

In one embodiment, a therapeutic hydrogel material is delivered to a stroke cavity, wound, or other damaged tissue and includes heparin nanoparticles physically entrained within the hydrogel matrix. The heparin nanoparticles are not bound to, immobilized to, or complexed with any other growth factor or the like at the time of delivery or administration. The heparin nanoparticles are bare or naked nanoparticles. In one embodiment, the therapeutic hydrogel material is an in situ gelling hyaluronic acid-based hydrogel that contains a plurality of heparin nanoparticles distributed within the hydrogel matrix. The hydrogel is crosslinked in one embodiment with a biodegradable crosslinker such as a matrix metalloproteinase (MMP) labile peptide as the crosslinker, resulting in a hydrogel that is both hyaluronidase degradable and MMP degradable. The hydrogel material may also be optionally modified with a cell adhesion peptide such as RGD derived from fibronectin to allow for integrin-mediated cell attachment to the hydrogel scaffold.

The therapeutic hydrogel material with the heparin nanoparticles reduces inflammation and promotes tissue repair through the generation of vascular and axonal networks within the wound. In some embodiments, the therapeutic hydrogel material promotes tissue ingrowth (i.e., tissue growth). As noted, the therapeutic hydrogel material may also promote the formation of axons and vessels. Importantly, the heparin nanoparticles used herein do not exhibit blood thinning properties (i.e., they do not act as a blood thinner) which are present in polymeric heparin. The heparin nanoparticles thus avoid the complications associated with the use of conventional heparin that has natural blood thinning properties. The therapeutic hydrogel material described herein further exhibits reduced damaged-induced scar thickness and reduced inflammatory response for wounds and other damaged tissue.

In some embodiments, the heparin nanoparticle-containing hydrogel material may be injected directly within a stroke cavity. In other embodiments, the heparin nanoparticle-containing hydrogel material is delivered to a wound site (e.g., chronic wound) or other damaged tissue. Types of tissue that may be used with the heparin nanoparticle-containing hydrogel material includes skin as well as other tissue. In one embodiment, the heparin nanoparticle-containing hydrogel material may be co-delivered to the stroke cavity, wound site, or other damaged tissue with a crosslinker that crosslinks the hydrogel in situ. In other embodiments, crosslinking may be performed by the use of a crosslinker along with a photoinitiator that initiates the crosslinking process in response to applied light or radiation.

In one aspect of the invention, a therapeutic hydrogel material is provided that includes an in situ gelling hydrogel material that incorporates naked heparin nanoparticles therein. There is no need for immobilizing any growth factor such as VEGF to the heparin nanoparticles as the naked heparin nanoparticles themselves provide the therapeutic benefits. In one preferred embodiment of the invention, the hydrogel is a hyaluronic acid-based hydrogel (e.g., hyaluronic acid functionalized with acrylamide groups). A biodegradable crosslinker (e.g., a matrix metalloproteinase (MMP) labile peptide) is used with the therapeutic hydrogel material to form the crosslinked hydrogel in situ within the stroke cavity, wound, or other damaged tissue of a mammalian subject. The therapeutic hydrogel material includes, in some optional embodiments, a cell adhesion peptide.

In another aspect of the invention, during clinical use for stroke patients, the patient or subject (e.g., human or other mammalian subject) will typically be first given a scan such as a magnetic resonance imaging (MRI) scan to localize the location and volume of the stroke site. The first three days (e.g., at about five days) after stroke are associated with a massive inflammatory response where cellular debris resulting from cell death in the damaged site are cleared by specialized inflammatory cells (microphages/microglia) leaving behind an empty cavity. In addition, astrocytes migrate to the border of the stroke site and undergo an extensive morphology remodeling and extend processes around the lesion to form a scar that compartmentalizes the degraded tissue in order to limit inflammation to the boundaries of the stroke. This astrocytic scar becomes a physical barrier to tissue infiltration and growth within the wound. The specific localization of both the infarct (stroke cavity) and the pen-infarct areas are determined with three-dimensional intra-cerebral coordinates (x, y and z). To access the stroke cavity, a hole or access passageway is drilled in the subject's skull (e.g., craniotomy) adjacent to the site of the stroke. Most strokes occur in the cerebral cortex or outer layer of brain tissue which can be then be readily accessed after the formation of the craniotomy. A delivery device, which may be a syringe or the like that contains the injectable therapeutic hydrogel material described herein, is then inserted into the craniotomy and the therapeutic material is then delivered to the stroke cavity. The therapeutic hydrogel material is loaded in the syringe as a liquid and solidifies (or gels) in situ within the stroke cavity to form a gelatinous solid with similar mechanical properties to the brain. Once in place, the hydrogel material provides the therapeutic benefits. Notably, the therapeutic hydrogel material may provide therapeutic benefits even though administered days after the stroke onset.

In another embodiment, the therapeutic hydrogel material may be applied to brain tissue. For example, the therapeutic hydrogel material may be applied to brain tissue and crosslinked in situ.

The therapeutic hydrogel material may also be delivered directly to a wound site or other damaged tissue of a mammalian subject. The therapeutic hydrogel material may be delivered with or without the aid of a delivery device. The wound site may be located on skin or epidermal tissue however it should be appreciated that the therapeutic hydrogel material may be applied to other organs and/or tissue types. For example, the therapeutic hydrogel material may be applied to skin tissue.

In another embodiment, a kit may be provided that includes a hyaluronic acid-based hydrogel precursor solution containing a plurality of naked heparin nanoparticles and a biodegradable crosslinker for crosslinking the hyaluronic acid-based hydrogel precursor solution into a crosslinked hydrogel. The kit may also include a delivery device such as, for example, a syringe, tube(s) or the like. In other embodiments, the crosslinker and/or hyaluronic acid-based hydrogel precursor may further comprise a photoinitiator and kit may include a light source that is used to illuminate the mixture to crosslink the hydrogel in situ. The mixture may also be applied manually using, for example, the hands or fingers.

### BRIEF DESCRIPTION OF THE DRAWINGS

FIG. 1 illustrates a cross-sectional view of a mammalian brain showing a stroke cavity. An injectable therapeutic hydrogel material is being delivered to the stroke cavity via a delivery device.

FIG. 2 illustrates a schematic representation of a stroke cavity that contains a crosslinked hydrogel that forms the therapeutic hydrogel material according to one embodiment.

FIG. 3 illustrates a sequence of operations or flowchart that outlines a method of making the therapeutic hydrogel material that is delivered to the stroke cavity (or other delivery site).

FIG. 4A illustrates fluorescent images of vessels (Glut-1) with nuclei marker Dapi (blue), a marker of proliferation (BrdU), and pericyte/smooth muscle cells (PDGFR-B) in and around the stroke site (*) at day 10 after gel transplantation (2 weeks after stroke). Single dashed line indicate border between infarct/peri-infarct. Asterisk (*) represents infarct site.

FIG. 4B illustrates the quantification of the vascular area (% Glut-1 area) in the infarct.

FIG. 4C illustrates quantification of angiogenesis (Glut-1/BrdU number of cell) in the infarct.

FIG. 4D illustrates quantification of pericyte vascular coverage (% PDGFR-β area) in the infarct area.

FIG. 4E illustrates the quantification of the vascular area (% Glut-1 area) in the peri-infract area.

FIG. 4F illustrates quantification of angiogenesis (Glut-1/BrdU number of cell) in the pen-infarct area.

FIG. 4G illustrates quantification of pericyte vascular coverage (% PDGFR-β area) in the pen-infarct area. For FIGS. 4A-4G No gel=stroke only condition, empty gel=HA hydrogel, gel+nH=HA hydrogel with 1 μg heparin nanoparticles (nH). Data is presented using a min to max box plot. Each dot in the plots represents one animal and p values were determined by One-way ANOVA with a Tukey's post-hoc test, with ** and **** indicating p<0.01 and p<0.0001, respectively. Scale bar: 100 μm.

FIG. 5A illustrates fluorescent images of vessels (Glut-1, red), Angiopoietin-2 (green) and the nuclei marker Dapi (Blue) in the pen-infarct area in the different conditions at day 10 after gel transplantation (2 weeks after stroke).

FIG. 5B illustrates a graph showing the quantification of the positive area for Angiopoetin-2 in the pen-infarct area. No gel=stroke only condition, empty gel=HA hydrogel, gel+nH=HA hydrogel with 1 μg unloaded nH. Data is presented using a min to max box plot. Each dot in the plots represent one animal and p values were determined by One-way ANOVA with a Tukey's post-hoc test, with * indicating P<0.05. Scale bar: 50 μm.

FIG. 6A illustrates fluorescent images of neuroblasts (Dcx), the proliferation marker BrdU, and the nuclei marker Dapi at day 10 after gel transplantation (2 weeks after stroke). Single dashed line indicate border between infarct/peri-infarct. Asterisk (*) represents infarct site.

FIG. 6B illustrates a graph showing the quantification of the total number of neuroblasts (Dcx) in the ipsilateral ventricle.

FIG. 6C illustrates a graph showing the quantification of the total number of neuroblasts migrating from the ipsilateral ventricle to the stroke area.

FIG. 6D illustrates a graph showing the quantification of axonal neurofilaments (NF200) area in the peri-infarct area.

FIG. 6E illustrates a graph showing the angle of penetration of NF200 neurofilament into the infarct area. For FIGS. 6A-6E No gel=stroke only condition, empty gel=HA hydrogel, gel+nH=HA hydrogel with 1 μg unloaded nH. Data is presented using a min to max box plot. Each dot in the plots represents one animal and p values were determined by One-way ANOVA with a Tukey's post-hoc test, with * indicating p<0.05. Scale bar: 100 μm.

FIG. 7A illustrates fluorescent images of microglia (Iba1) and the astrocytic scar (GFAP) with the nuclei marker Dapi at day 10 after gel transplantation (2 weeks after stroke). Single dashed line indicate border between infarct/peri-infarct. Asterisk (*) represents infarct site. Double dashed line indicates thickness of scar. Note the thinner scar in the Gel+nH experiments.

FIG. 7B illustrates quantification of the microglial area (Iba1) in the infarct area.

FIG. 7C illustrates measurement data of the of the astrocytic scar thickness.

FIG. 7D illustrates quantification of the microglial area (Iba1) in the peri-infarct area.

FIG. 7E illustrates a graph showing the quantification of brain levels of the cytokine TNF-alpha in the infarct area. For FIGS. 7A-7E No gel=stroke only condition, empty gel=HA hydrogel, gel+nH=HA hydrogel with 1 μg unloaded nH. Data is presented using a min to max box plot. Each dot in the plots represents one animal and p values were determined by One-way ANOVA with a Tukey's post-hoc test, with *, ** indicating p<0.05 and p<0.01, respectively. Scale bar: 100 μm.

FIG. 8 illustrates a graph illustrating the quantification of the anti-coagulant properties of heparin nanoparticles using the mouse tail vein bleeding assay where the bleeding time was measured in seconds after intravenous injection with saline (PBS), 2 μg of heparin or heparin nanoparticles (nH). Data is presented using a min to max box plot. Each dot in the plots represent one animal and p values were determined by One-way ANOVA with a Tukey's post-hoc test, with **** indicating P<0.0001.

FIG. 9A illustrates the quantification of the blood-brain barrier opening at day 10 after gel transplantation (2 weeks after stroke) in mice injected with empty gel and gel+nH compared with no gel injection 5 days after stroke.

FIG. 9B illustrates the measurement of the infarct volume at day 10 after gel transplantation (2 weeks after stroke) in mice injected with empty gel and gel+nH compared with no gel injection 5 days after stroke.

FIG. 9C illustrates the measurement of the ipsilateral cortex at day 10 after gel transplantation (2 weeks after stroke).

FIG. 9D illustrates the measurement of the ipsilateral hemisphere volume (ratio with the contralateral side) at day 10 after gel transplantation (2 weeks after stroke). For FIGS. 9A-9D No gel=stroke only condition, empty gel=HA hydrogel, gel+nH=HA hydrogel with 1 μg unloaded nH. Data is presented using a min to max box plot. Each dot in the plots represents one animal and p values were determined by One-way ANOVA with a Tukey's post-hoc test.

### DETAILED DESCRIPTION OF ILLUSTRATED EMBODIMENTS

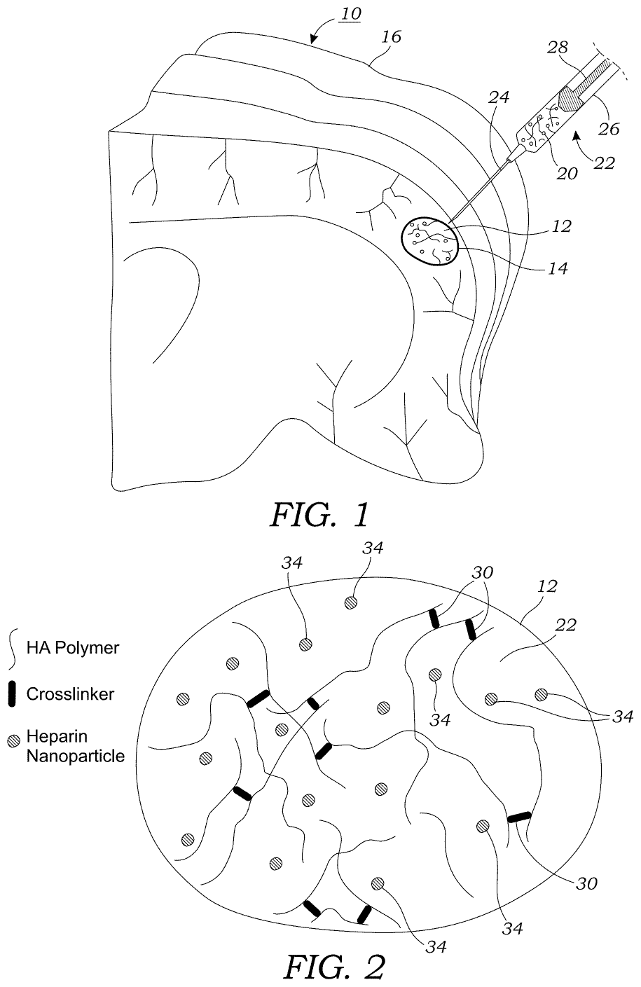

FIG. 1 illustrates a cross-sectional view of a mammalian brain 10 that includes stroke cavity 12 formed therein. As explained herein, in one embodiment the delivery site is a stroke cavity 12 such as that illustrated in FIG. 1 that naturally forms after stroke. In other embodiments, however, the delivery site is a wound site or site of damaged tissue. This may include epidermal tissue or skin, or other organ/body tissue. Thus, the illustration of FIG. 1 for treatment of stroke is but one application of the therapeutic hydrogel material 20. Further, the therapeutic hydrogel material 20 may be applied topically to other tissue types as discussed herein.

With respect to the treatment of stroke, after the initial cell death that follows a stroke, the clearance of debris in the lesion leaves a compartmentalized cavity 12 that can accept a large volume of the therapeutic hydrogel material 20 described herein without further damaging the surrounding healthy parenchyma. This stroke cavity 12 is situated directly adjacent to the pen-infarct tissue area 14, the region of the brain that undergoes the most substantial repair and recovery, meaning that any therapeutic delivered to the cavity 12 will have direct access to the tissue target for repair. In addition to being deliverable to the stroke cavity 12 via injection, the therapeutic hydrogel material 20 may also be transplanted in the pen-infarct area 14, or the brain surface 16. In other embodiments, the therapeutic hydrogel material may be applied with an applicator or even manually.

FIG. 1 further illustrates a delivery device 22 that is used to deliver the therapeutic material 20 to the stroke cavity 12. As seen in FIG. 1, the delivery device 22 is in the form of a syringe that includes a needle 24 and barrel 26 that holds the injectable therapeutic hydrogel material 20. A depressor 28 is used to eject the therapeutic hydrogel material 20 from the end of the needle 24 and into the stroke cavity 12. During clinical use, the patient or subject (e.g., human or other mammalian subject) will typically be first given a scan such as a magnetic resonance imaging (MRI) scan to localize the location and volume of the stroke cavity 12. The first three days (e.g., at about five days) after stroke are associated with a massive inflammatory response where cellular debris resulting from cell death in the damaged site are cleared by specialized inflammatory cells (microphages/microglia) leaving behind an empty cavity. In one embodiment, the therapeutic hydrogel material 20 is preferably injected within fifteen (15) days of stroke onset and after day three (3) post-stroke to avoid the severe post-stroke inflammation and edema in the damaged brain. It should be appreciated, however, that in other uses the therapeutic hydrogel material 20 beyond these specific ranges.

The specific localization of both the infarct (stroke cavity 12) and the pen-infarct areas are determined with three-dimensional intra-cerebral coordinates (x, y and z). While a syringe is illustrated as the delivery device 22 the therapeutic hydrogel material 20 may also be delivered using a catheter-based device or the like to deliver the injectable therapeutic hydrogel material 20 from a location outside the subject's brain to the stroke cavity 12.

To access the stroke cavity, a hole or access passageway is drilled in the subject's skull (e.g., craniotomy) adjacent to the site of the stroke. Most strokes occur in the cerebral cortex or outer layer of brain tissue which can be then be readily accessed after the formation of the craniotomy. The delivery device 22, which may be a syringe or the like as described above that contains the therapeutic hydrogel material 20, is then inserted into the craniotomy and the therapeutic hydrogel material 20 is then delivered to the stroke cavity 12. The therapeutic hydrogel material 20 then crosslinks or gels within the stroke cavity 12 and provides the therapeutic benefits. In some embodiments, the volume of therapeutic hydrogel material 20 that is delivered substantially fills the stroke cavity 12. Crosslinking of the therapeutic hydrogel material 20 may be accomplished by the addition of a crosslinking agent just prior to delivery. In one alternative, crosslinking may be accomplished by co-delivering the therapeutic hydrogel material 20 and the crosslinking agent. For example, the delivery device 22 may include separate compartments that contain the therapeutic hydrogel material 20 and the crosslinking agent which are then mixed upon delivery from the delivery device 22. In yet another alternative, the crosslinking may be initiated by the use of photoinitiator along with a crosslinking agent that crosslinks in response to applied light (e.g., ultraviolet light). For example, Eosin photoinitiators are known to be used for photopolymerization of hydrogels.

Notably, the therapeutic hydrogel material 20 may provide therapeutic benefits even though administered days after the stroke onset. The delivery device 22 may be manually or automatically controlled to dispense the therapeutic hydrogel material 20 into the stroke cavity 12. For example, the delivery device 22 may be mounted on a robotic arm or the like that can be used to precisely place the tip of the needle 24 within the stroke cavity 12 using surgical robotic techniques known to those skilled in the art.

FIG. 2 illustrates the injectable therapeutic hydrogel material 20 that has gelled in situ within the stroke cavity 12. In one embodiment, the therapeutic hydrogel material 20 is formed from a hyaluronic acid-based hydrogel that forms an amorphous non-fibrous hydrogel composed of hyaluronic acid, which has been shown to promote neural differentiation, angiogenesis and axonogenesis. As explained herein, the hyaluronic acid is functionalized with acrylamide functionality (HA-AC) because its kinetics are slower than those of acrylates or vinyl sulfones, which allowed for enough time for injection and ensure that the entire stroke cavity 12 was full of gel before complete crosslinking. The therapeutic hydrogel material 20 precursor remains liquid for a period after mixing, such that it can be injected into the brain 10 through a minimally invasive needle 24; and will gel within the stroke cavity 12, conforming to the boundaries of this damaged brain tissue. The mechanical properties of this injectable therapeutic hydrogel material 20 are similar to those of normal brain. While a hyaluronic acid-based hydrogel material 20 is described herein should be appreciated that other hydrated hydrogels or polymers may be used including, for example, poly(ethylene glycol) or PEG-based hydrogels, Poly(2-hydroxyethyl methacrylate) (PolyHEMA), alginate, chitosan, and dextran.

As seen in FIG. 2, the therapeutic hydrogel material 20 crosslinks or gels via the crosslinker 30. In one preferred embodiment, the crosslinker 30 is a biodegradable crosslinker 30. For example, the crosslinker 30 may include a matrix metalloproteinase (MMP) labile or degradable peptide. An example, of such an MMP labile peptide includes (Ac-GCREGPQGIWGQERCG-NH2, MMP-degradable [SEQ ID NO: 1]. In stroke, local production of hyaluronidases and matrix metalloproteases modify the tissue environment and can be coopted to alter the duration of effect of an injectable material. Thus, in the therapeutic hydrogel material 20 that uses a degradable peptide, the resulting hydrogel is both hyaluronidase degradable and MMP degradable, and is designed with a stiffness corresponding to the brain to reduce the local inflammatory response. In other embodiments, the crosslinker 30 may be non-degradable. An example of a non-degradable crosslinker includes Ac-GCREGDQGIAGFERCG-NH2 [SEQ ID NO: 2].

FIG. 2 also illustrates the therapeutic hydrogel material 20 that includes a plurality of heparin nanoparticles 34 (also referred to in some instances as “nH” herein) that are well dispersed within the hydrogel. The term “nanoparticles” refers to small nanometer-sized particles of heparin and in particular heparin nanoparticles that have a diameter or width within the range of about 200 nm to less than 1 μm. The nanoparticles of heparin 34 are formed using an inverse emulsion polymerization process that generates spherically-shaped nanoparticles of heparin. The diameter of the heparin nanoparticles 34 is, in one embodiment, generally within the range of about 90 nm to about 200 nm when contained in water or aqueous-based fluid. This range includes all values between the lower and upper limits (e.g., 91 nm, 92 nm, 93 nm . . . 199 nm). It should be appreciated, however, that the therapeutic hydrogel material 20 may still have therapeutic effects for heparin nanoparticles 34 that fall outside the specific diameter range cited above. The nanoparticles of heparin 34 are entrained within the porous structure of the hydrogel. That is to say, the nanoparticles of heparin 34 are physically retained inside the porous hydrogel structure and are not covalently bound to the hydrogel scaffold. The hydrogel generally has a pore size between 20 nm to 300 nm and the nanoparticles of heparin 34 are distributed and retained within the larger hydrogel scaffold superstructure. Even when the diameter of the nanoparticle of heparin 34 is less than the pore size of the hydrogel, the nanoparticle of heparin 34 is nonetheless entrained within the tortuous pores of the hydrogel scaffold.

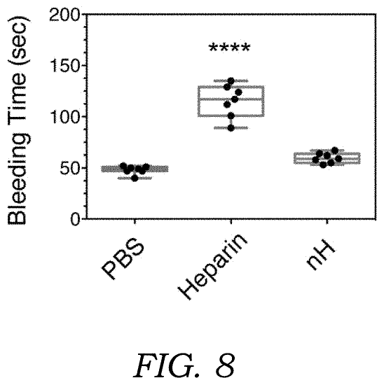

The heparin nanoparticles 34, when delivered as part of the therapeutic hydrogel material 20, do not contain any other molecules or moieties bound thereto as they are “naked.” The heparin nanoparticles 34 are, however, designed such that they retained their ability to bind growth factors and cytokines, but not the native heparin ability to reduce blood coagulation (see FIG. 8), such that heparin the nanoparticles 34 could sequester and retain endogenously expressed heparin binding signals after stroke. Importantly, the heparin nanoparticles 34 lack the blood thinning properties and leads to complications when using other types of heparin (e.g., polymeric heparin). Thus, while the heparin nanoparticles 34 are delivered in a “naked” state, post-delivery the heparin nanoparticles 34 may immobilize and retain various endogenous growth factors and/or cytokines. This include, for example, interleukin 4 (IL4) and interleukin 10 (IL10). The therapeutic hydrogel material 20 that incorporates the naked heparin nanoparticles 34 is advantageous because: (1) the naked heparin nanoparticles 34 do not exhibit blood thinning; (2) the naked heparin nanoparticles 34 retain the ability to bind growth factors and/or cytokines; and (3) the therapeutic hydrogel material 20 reduces localized inflammation.

It was found that through engineering a heparin nanoparticle-containing therapeutic hydrogel material 20 and injecting it directly within the stroke cavity 12 induced the formation of new vascular and neuronal structures. The formation of a robust, mature and highly developed vascular bed within the stroke cavity 12 helps develop and pattern the nervous system. The formation of vascular bed enhances the migration of immature neurons.

The therapeutic hydrogel material 20 may also optionally include cell adhesion peptides. For example, the hyaluronic acid-based hydrogel may be functionalized with a cell adhesion peptide. As example of an adhesion peptide includes fibronectin-derived RGD adhesion peptide Ac-GCGYGRGDSPG-NH2 [SEQ ID NO: 3] (RGD, Genscript, Piscataway, N.J.). As explained herein, during formation of the therapeutic hydrogel material 20 the RGD peptide may be clustered in the hyaluronic acid-based hydrogel. This may be accomplished by crosslinking of a smaller sub-volume (e.g., around 15%) of the hyaluronic acid precursor (HA-AC) material followed by the addition of RGD-free hyaluronic acid precursor material (e.g., around 85%).

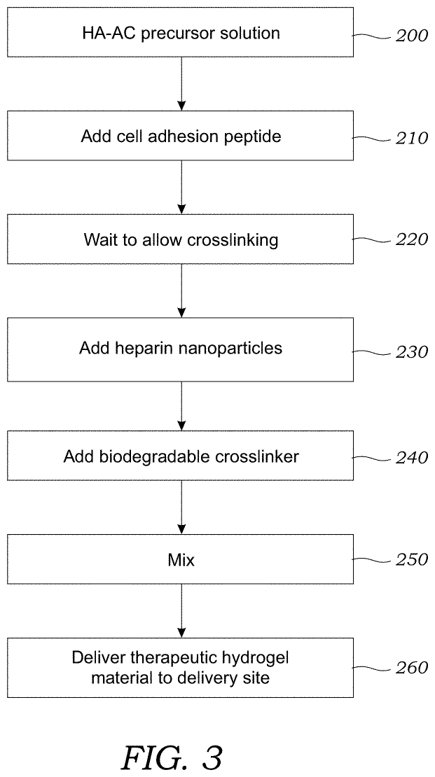

FIG. 3 illustrates a flowchart of operations used to generate the therapeutic hydrogel material 20. As seen in FIG. 3 in operation 200, the precursor solution of HA-AC is formed. For example, this precursor solution may be made by dissolving lyophilized HA-AC in 0.3 M HEPES buffer for 15 minutes at 37° C. Next, as seen in operation 210, the optional cell adhesion peptide (e.g., RGD peptide) is added to this solution. This operation or step may be omitted in some embodiments. As described above, it is preferably to create clusters of RGD peptide within the precursor solution of HA-AC. This may be accomplished by adding the RGD peptide to a smaller sub-volume (e.g., around 15%) of the hyaluronic acid precursor (HA-AC) material to obtain a degree of clustering of around 1.17, and reacted at room temperature for around 10-20 minutes to allow for crosslinking (waiting operation 220). This may be followed by the addition of the remaining RGD-free hyaluronic acid precursor material (e.g., around 85%). In operation 230, the heparin nanoparticles 34 are added to this solution. As seen in operation 240, prior to delivery to the stroke cavity 12 the crosslinker 30 is added (e.g., the crosslinker 30 is co-delivered with the hydrogel precursor solution). This may include a small aliquot of the MMP-degradable (or non-degradable) crosslinker 30 dissolved in an appropriate buffer solution (e.g., 0.3 M HEPES). Next, in operation 250, the mixture is well mixed and loaded into the optional delivery device 22. As seen in operation 260, the therapeutic hydrogel material 20 is then delivered to the delivery site (e.g., stroke cavity 12 with the delivery device 22). The therapeutic hydrogel material 20 may also be delivered to a wound site (or other damaged tissue) using an applicator, delivery device, or even manually.

While FIG. 3 illustrates an embodiment where the therapeutic hydrogel material 20 crosslinks upon mixture (operations 250) it should be understood that in other embodiments, a stimulus may need to be provided to initiate crosslinking. For example, photopolymerization may be used with a crosslinker and photoinitiator (e.g., Irgacure® photoinitiator) and a light emitting device (e.g., UV emitting device) to form the in situ therapeutic hydrogel material 20.

In some embodiments, the therapeutic hydrogel material 20 may be provided as part of a kit. For example, the kit may include a hyaluronic acid-based hydrogel precursor solution containing the plurality of naked heparin nanoparticles 34. The kit may also contain a biodegradable crosslinker 30 for crosslinking the hyaluronic acid-based hydrogel precursor solution into a crosslinked hydrogel. The kit may also include, in embodiments where the therapeutic hydrogel material 20 is crosslinked using photopolymerization a photoinitiator. The crosslinker 30 may be provided in a separate vial or container which can be added just prior to delivery. In other embodiments where, for example, photopolymerization is used, the crosslinker may be contained separate or even in the hyaluronic acid-based hydrogel precursor solution in an opaque container that avoids exposure to light. The kit may also include a cell adhesive peptide such as RGD. The adhesive peptide may also be provided in a separate vial or container that is added to the hyaluronic acid-based hydrogel precursor solution as explained herein. The kit may also include in some embodiments, the delivery device 22. This may include a syringe, tube(s), mixing device, or other applicator. Alternatively, the operating room may use an existing delivery device 22 which is loaded with solutions as part of the kit.

### Experimental

An in situ gelling therapeutic hydrogel material was selected as the platform for the results presented herein. The therapeutic hydrogel material is a hyaluronic acid hydrogel based on thiol-acrylamide Michael-type addition as described herein with a MMP labile peptide used as the crosslinker which resulted in a hydrogel that is both hyaluronidase degradable and MMP degradable, designed with a stiffness corresponding to the brain to reduce the local inflammatory response.

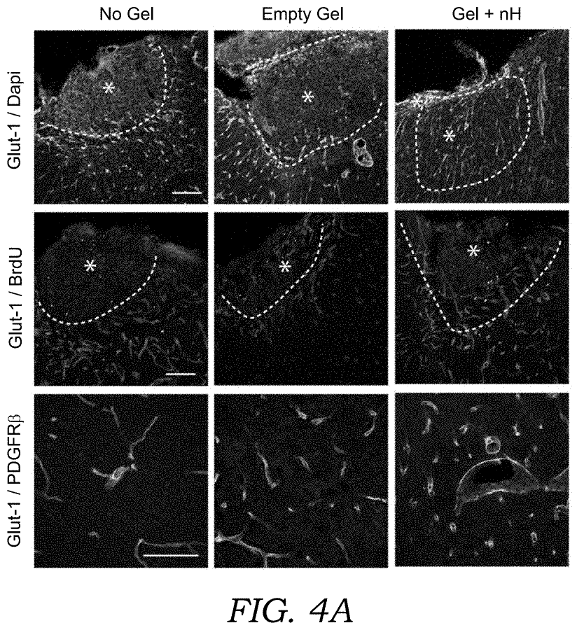

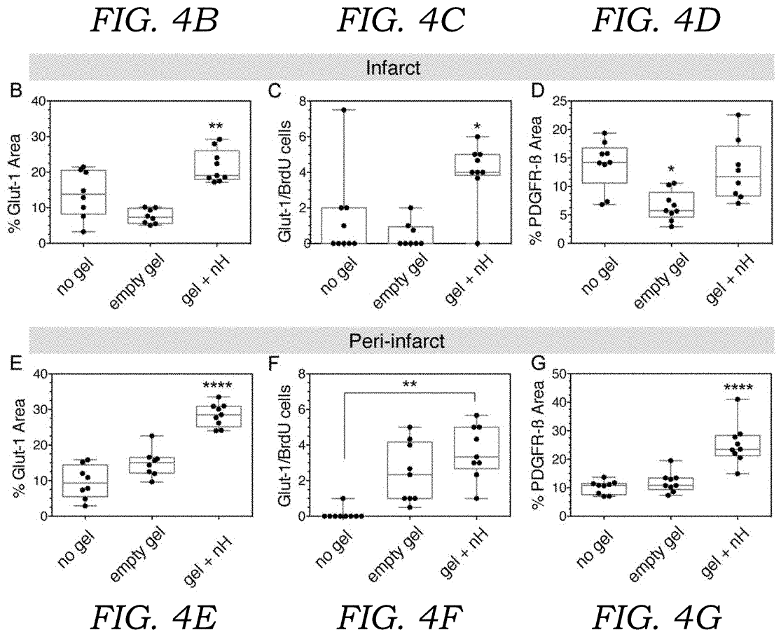

FIGS. 4A-4G illustrate the results of post-stroke response and vascular remodeling. The addition of nH to the HA hydrogel significantly increases the vascular area in both the stroke site and the surrounding tissue (peri-infarct) compared with the no gel and empty gel groups (FIG. 4B and FIG. 4D). This result is associated with an increased number of double-labeled BrdU/Glut-1 cells in the infarct and peri-infarct area (FIG. 4C and FIG. 4F) indicating a greater number of proliferating endothelial cells, characteristic to angiogenesis and vessel growth. The quantification of pericytes along vessels shows that the absence of heparin nanoparticles in the hydrogel significantly decreases pericyte coverage in the infarct (FIG. 4D), and that the addition of heparin particles to the gel significantly increases pericyte coverage in the peri-infarct area (FIG. 4G).

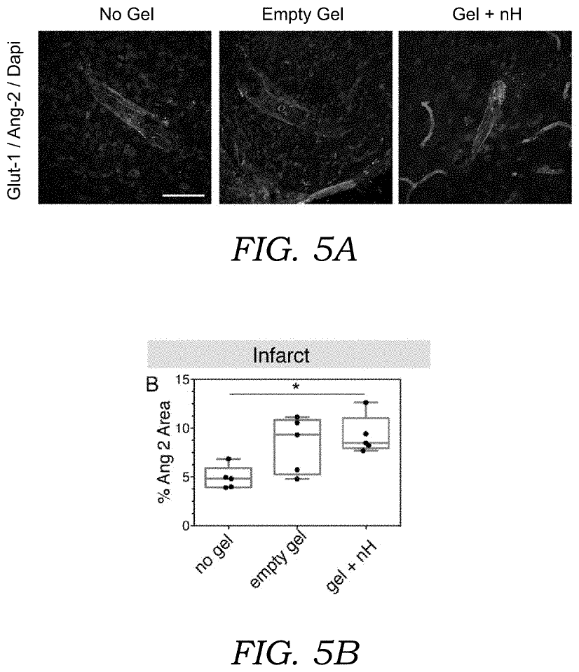

FIG. 5A illustrates fluorescent images of vessels (Glut-1), Angiopoietin-2, and the nuclei marker Dapi in the peri-infarct area in the different conditions at day 10 after gel transplantation (2 weeks after stroke). As seen in FIG. 5B, the addition of heparin nanoparticles to the hydrogel significantly increases the secretion of Angiopoietin-2, known to play a distinct role in angiogenesis and in coupling of angiogenesis to other elements of tissue repair, in the infarct area compared with the No gel group.

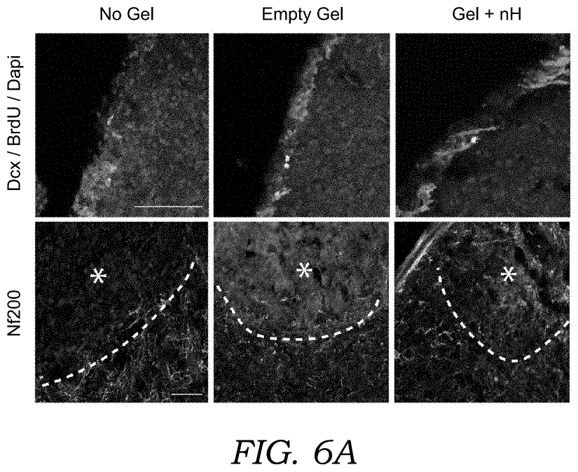

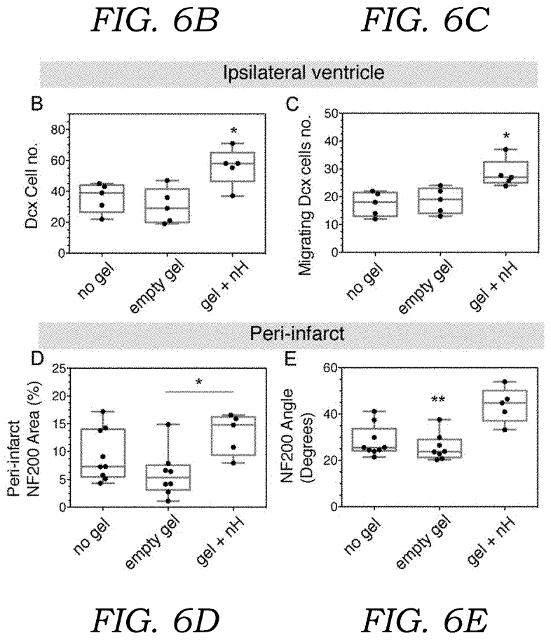

FIGS. 6A-6E illustrate the results of post-stroke neurogenesis and axonal sprouting. The addition of heparin nanoparticles to the hydrogel increases significantly the total number of neuroblast (neural progenitor cell, Dcx) located along the ipsilateral ventricle compared with the no gel and empty gel groups (FIG. 6B), as well as the total number of neuroblast migrating towards the infarct area (FIG. 6C). The brain administration of gel+nH increases significantly the surface of axons present around the infarct compared with the empty gel group (FIG. 6D), which is characteristic of axonal sprouting towards the site of stroke. The absence of nH in the gel reduces significantly the angle (FIG. 6E) at which these new axons penetrate the site of injury compared with gel+nH (E) which represent a loss of physiological phenotype in axonal growth where axons penetrate a regenerating tissue at an angle of 90°.

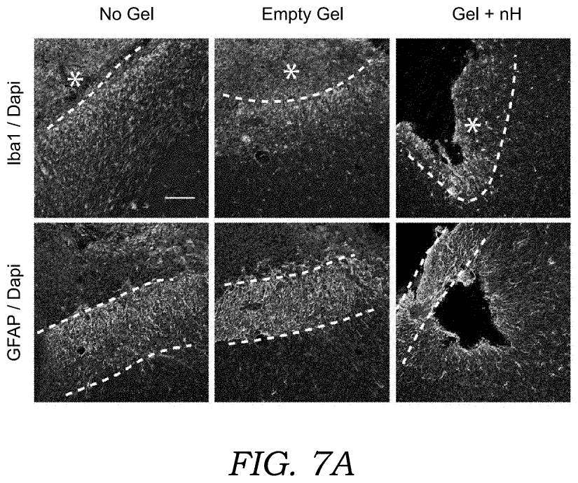

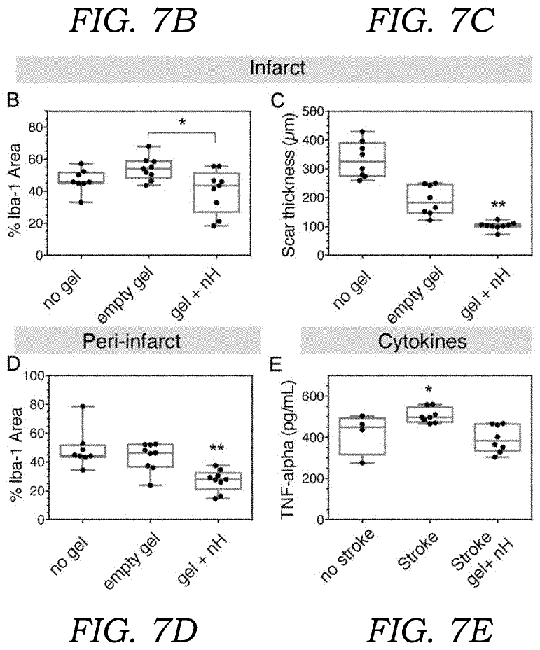

FIGS. 7A-7E illustrate the role of heparin nanoparticles in post-stroke inflammation. The addition of heparin nanoparticles to the hydrogel significantly reduces the inflammatory response by reducing the surface of the inflammatory cells, microglia inside and around the stroke site (FIG. 7B and FIG. 7D). In addition, gel+nH reduces significantly the thickness of the astrocytic scar, known to be correlated to the intensity of the post-stroke inflammatory response around the site of damage (FIG. 7C). A measurement of brain cytokine levels before and after stroke reveals that stroke induces a significant increase of the pro-inflammatory cytokine TNF-alpha in the site of damage. The injection of a gel+nH in the stroke brain is associated with brain levels of TNF-alpha that are similar to the pre-stroke (healthy brain) condition (FIG. 7E).

FIG. 8 illustrates the anti-coagulant properties of the heparin nanoparticles. Mice were submitted to a lesion on the tail to induce bleeding. The injection of heparin alone shows a significant increase of the bleeding time, which confirms the anti-coagulant effect of heparin. The injection of heparin nanoparticles however does not increase the bleeding time, showing that the chemical and structural modification of heparin to form nanoparticles prevents heparin from exerting anti-coagulant properties.

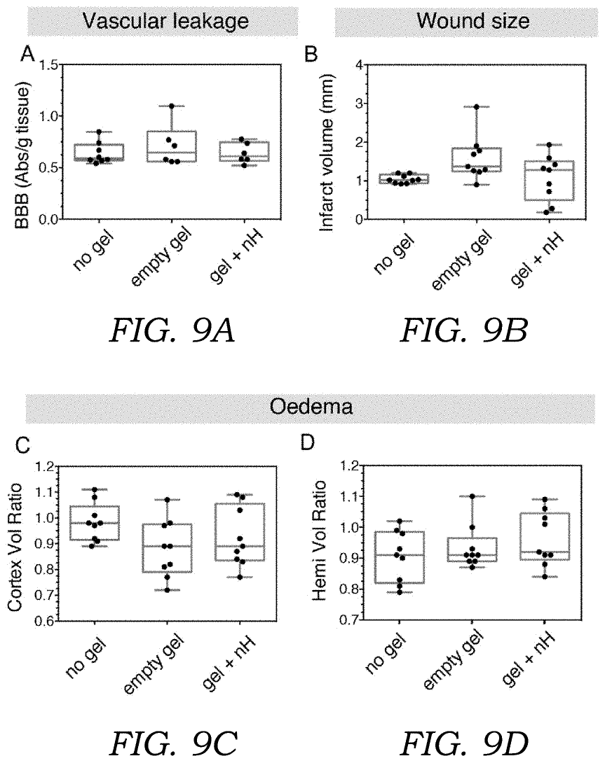

FIGS. 9A-9D illustrate the absence of secondary effects. The brain administration of heparin nanoparticles in HA hydrogel does not increase vascular leakage (FIG. 9A), the wound size (FIG. 9B), or edema in either the cortex where the gel was injected (FIG. 9C) or in the rest of the hemisphere (FIG. 9D).

Materials and Methods

Heparin Nanoparticle Synthesis

Heparin was first modified with p-azidobenzyl hydrazide (ABH) through 1-ethyl-3-(3-dimethylaminopropyl)carbodiimide (EDC) mediated conjugation in a 1:3 molar ratio of ABH to available carboxylic acids at pH 5.5 in a 100 mM solution of 2-(N-morpholino) ethanesulfonic acid (MES) buffer. The remaining carboxylic acid groups on heparin were then conjugated with N-(3-Aminopropyl) methacrylamide in 27 molar excess through EDC coupling chemistry overnight at room temperature in MES buffer. The solution was then dialyzed against distilled (DI) water and lyophilized for two days. The final product was dissolved in a 100 mg/ml solution of sodium acetate at pH 4, then combined with Tween-80 and Span-80 (8% HLB) and sonicated to form nanoparticles. The radical polymerization was initiated by mixing heparin in a ten-fold volume of hexane combined with N,N,N′,N′-tetramethyl-ethane-1,2-diamine (TEMED) and ammonium persulfate (APS). The resultant nanoparticles were purified using liquid-liquid extraction in hexane and bubbling nitrogen gas was used to evaporate off the excess of hexane. The nanoparticles were then dialyzed in 100 kD MWCO dialysis units for 12 hours and stored at +4 C. The amount of heparin in the solution was determined by lyophilizing a small aliquot of the solution.

Heparin Nanoparticle Characterization

Dynamic Light Scattering (DLS) was used to characterize the Z-average (diameter) and polydispersity index (PDI) of heparin nanoparticles after each preparation step. Samples were loaded into a filtered DI water quartz cuvette and analyzed by a Malvern Zetasizer where ten runs of three measurements each were performed. Transmission Electron Microscopy (TEM) with a T12 Quick CryoEM was performed in order to confirm the morphology and size distribution of nanoparticles: a drop of sample solution (1 mg/mL) was placed onto a 300 mesh copper grid coated with carbon. The nanoparticles were then negatively stained by 2 wt % photungstic acid (PTA) solution.

Tail Vein Bleeding Assay

Animal procedures were performed in accordance with the US National Institutes of Health Animal Protection Guidelines and approved by the Chancellor's Animal Research Committee as well as the UCLA Office of Environment Health and Safety. Briefly, under isoflurane anesthesia (2-2.5% in a 70% N₂O/30% O₂ mixture), young adult C57BL/6 male mice (8-12 weeks) obtained from Jackson Laboratories were placed on a warming pad and injected intravenously with heparin, heparin nanoparticles or saline (4 U/kg, 50 μl) 30 minutes before testing. A transverse incision was made with a scalpel over a lateral tail vein at a position where the diameter of the tail is 2.5 mm. The depth of the incision is designated to macerate the tail vein. The tail is then hung over the edge of a table and immersed in normal saline at 37° C. in a hand-held conical tube. The time (in sec) from the incision to the cessation of bleeding is recorded as the tail vein bleeding time.

Hyaluronic Acid Modification and Hydrogel Gelation

Hyaluronic acid (60,000 Da, Genzyme, Cambridge, Mass.) was functionalized with an acrylamide groups using a two-step synthesis as previously described in Lei, S. et al., The spreading, migration and proliferation of mouse mesenchymal stem cells cultured inside hyaluronic acid hydrogels, Biomaterials 32, 39-47 (2011) and P. Moshayedi et al., Systematic optimization of an engineered hydrogel allows for selective control of human neural stem cell survival and differentiation after transplantation in the stroke brain, Biomaterials 105, 145-155 (2016), which are incorporated herein by reference.

After dissolving the HA (2.0 g, 5.28 mmol) in water, it was reacted with adipic dihydrazide (ADH, 18.0 g, 105.5 mmol) in the presence of 1-ethyl-3-(dimethylaminopropyl) carbodiimide hydrochloride (EDC, 4.0 g, 20 mmol) overnight at a pH of 4.75. The hydrazide-modified hyaluronic acid (HA-ADH) was purified with decreasing amounts of NaCl (100, 75, 50, 25 mmol) for 4 hours each via dialysis (8,000 MWCO). The solution was then purified via dialysis (8000 MWCO) in deionized water for 2 days. After 2 days purifying against deionized water, the HA-ADH was lyophilized. The HA-ADH was re-suspended in 4-(2-hydroxyethyl)-1-piperazine ethane-sulfonic acid (HEPES) buffer (10 mM HEPES, 150 mM NaCl, 10 mM EDTA, pH 7.4) and reacted with N-acryloxysuccinimide (NHS-AC), 1.33 g, 4.4 mmol) overnight. After purification via dialysis as described earlier, the HA-acrylamide (HA-AC) was lyophilized. The product was analyzed with 1H NMR (D₂O) and the degree of acrylamide modification (14.9%) determined by dividing the multiplet peak at δ=6.2 (cis and trans acrylate hydrogens) by the singlet peak at δ=1.6 (singlet peak of acetyl methyl protons in HA).

This hydrogel was chosen because of its biocompatibility with human tissue, as it is constituted of naturally occurring brain extracellular matrix constituents. The acrylamide functionality was used because its kinetics are slower than those of acrylates or vinyl sulfones, which allowed for enough time for injection and ensure that the entire stroke cavity was full of gel before complete crosslinking. The gel precursor remains liquid for a period after mixing, such that it can be injected into the brain through a minimally invasive needle; and will gel within the stroke cavity, conforming to the boundaries of this damaged brain tissue. The mechanical properties of this hydrogel are similar to those of normal brain. Finally, HA has been shown to promote angiogenesis in a mouse model of skin wound healing. However, as noted herein, other hydrogels besides HA may be used for the therapeutic hydrogel material 20.

Gelation

The hydrogel was made by dissolving the lyophilized HA-AC in 0.3 M HEPES buffer for 15 minutes at 37° C. Studies with stroke mice contained 500 μM of the adhesion peptide Ac-GCGYGRGDSPG-NH2 [SEQ ID NO: 3] (RGD, Genscript, Piscataway, N.J.). It has been previously found that clustered bioactive signals such as the adhesion peptide RGD results in significant differences in cell behavior when encapsulated inside three-dimensional HA. The highest degree of cell spreading, integrin expression and proliferation of encapsulated mouse mesenchymal stem cells was obtained with a ratio of 1.17 mole of RGD-reacting HA for 1 mole of RGD. The RGD peptide was dissolved in 0.3 M HEPES and added to 16% of the total HA-AC required to obtain a degree of clustering of 1.17, and reacted for 20 minutes at room temperature before being added to the rest of non-RGD reacting HA-AC. A total of 1 μg of heparin nanoparticles was added to the gel precursor solution. To crosslink the gels, an aliquot of the desired crosslinker (Ac-GCREGPQGIWGQERCG-NH2 [SEQ ID NO: 1], MMP-degradable or Ac-GCREGDQGIAGFERCG-NH2 [SEQ ID NO: 2], MMP-nondegradable) was dissolved in 0.3 M HEPES and added to the gel precursor solution. For viability and animal injections, the precursor was loaded into the Hamilton syringe directly after mixing in the desired crosslinking peptide.

Animal Experiment Design

Animal procedures were performed in accordance with the US National Institutes of Health Animal Protection Guidelines and approved by the Chancellor's Animal Research Committee as well as the UCLA Office of Environment Health and Safety. The permanent and distal Middle cerebral artery occlusion (MCAo) model is one of the models that most closely simulate human ischemic stroke and its pen-infarct penumbra as approximately 70% of human infarcts originate from the MCA. The MCAo model is considered to be suitable for reproducing cell death, inflammation, and blood-brain barrier (BBB) damage after stroke and has therefore been used in the majority of studies that address post-stroke repair mechanisms such as neurogenesis and angiogenesis.

Middle Cerebral Artery Occlusion (MCAO) Stroke Model

Focal and permanent cortical stroke was induced by a middle cerebral artery occlusion (MCAo) on young adult C57BL/6 male mice (8-12 weeks) obtained from Jackson Laboratories. Briefly, under isoflurane anesthesia (2-2.5% in a 70% N₂O/30% O₂ mixture), a small craniotomy was performed over the left parietal cortex. One anterior branch of the distal middle cerebral artery was then exposed, electrocoagulated and cut. Bilateral jugular veins were clamped for 15 min. Body temperature was maintained at 36.9±0.4° C. with a heating pad throughout the operation. In this model, ischemic cellular damage is localized to somatosensory and motor cortex and was chosen because of the high re-vascularization process after stroke in this region.

Hydrogel and Heparin Nanoparticle Intracranial Transplantation

Five days following stroke surgery, 6 μL of RGD—functionalized HA hydrogel containing heparin nanoparticles was loaded into a 25 μL Hamilton syringe (Hamilton, Reno, Nev.) connected to a syringe pump. The solution was then injected in liquid form directly into the stroke cavity using a 30-gauge needle at stereotaxic coordinates 0.26 mm anterior/posterior (AP), 3 mm medial/lateral, and 1 mm dorsal/ventral (DV) for the MCAO-strokes mice and at 1.5 mm medial/lateral for PT-stroked mice at an infusion rate of 1 μL/min. The control group was injected with an empty RGD-functionalized gel (Empty). The needle was withdrawn from the mouse brain immediately after the injection was complete. Ten days following the hydrogel transplantation, animals were given the DNA synthesis marker 5-bromo-2′-deoxyuridine (BrdU, Sigma, St Louis, Mo.; 100 mg/kg in 0.9% NaCl) intraperitoneally 4 and 2 hours before euthanasia to assess cell proliferation. In all experiments, the researchers were blind to the treatment given to each animal.

Mouse Tissue Processing and Immunohistochemistry

At 2 weeks post-stroke (10 days after transplantation), mice were transcardially perfused with 0.1 M PBS followed by 40 mL of 4% (wt/vol) paraformaldehyde (PFA). After isolation, the brain was post-fixed in 4% PFA overnight, cryoprotected in 30% sucrose in phosphate buffer for 24 hours and frozen. Tangential cortical sections of 30 μm-thick were sliced using a cryostat and directly mounted on gelatin-subbed glass slides. Brain sections were then washed in PBS and permeabilized and blocked in 0.3% Triton and 10% Normal Donkey Serum before being immunohistochemically stained. Primary antibodies were as follows: Rabbit anti-Glucose Transporter 1 (Glut-1-) (1:200; Abcam, Cambridge, Mass.) for vascular Endothelial Cells; goat anti-Platelet-derived Growth Factor Receptor β (1:400; PDGF-Rβ, R&D Systems, Minneapolis, Minn.) for pericytes; goat anti-doublecortin (DCX) (C18, 1:500; Santa Cruz Biotechnology, Santa Cruz, Calif.) for sub-ventricular neural progenitor cells; rat anti-BrdU (1:300; Abcam, Cambridge, Mass.); rat anti-GFAP (1:400; Zymed, San Francisco, Calif.) for astrocytes; rabbit anti-microglial-specific ionized calcium binding adaptor molecule 1 (Iba-1) (1:250; Wako Pure Chemical Industries, Japan) for microglial cells; rabbit anti-Neurofilament 200 (NF200) (1:100; Sigma-Aldrich, St Louis, Mo.), Angiopoietin-2 (1:250; Abcam, Cambridge, Mass.). Primary antibodies were incubated overnight at +4° C. followed by fluorescently labeled secondary antibody (Molecular Probes, Cergy-Pontoise, France, 1:400) for 1 h at room temperature. Cell nuclei were then counterstained with the nuclear marker 4′, 6-diamidino-2-phenylindole (DAPI, 1:500, Invitrogen) for 10 minutes at room temperature. After 3×10 minute washes in PBS, the slides were dehydrated in ascending ethanol baths, and dewaxed in xylene and coverslipped over fluorescent mounting medium (Dako). Sections stained for BrdU were pretreated with 2N HCl for 30 min and neutralized with sodium borate buffer, pH 8.4, before incubation in primary antibody.

Microscopy and Morphoanalysis

Analyses were performed on microscope images of three coronal brain levels at +0.80 mm, −0.80 mm and −1.20 mm according to Bregma, which consistently contained the cortical infarct area. Each image represents a maximum intensity projection of 10 to 12 Z-stacks, 1 μm apart, captured at a 20× magnification with a Nikon C2 confocal microscope using the NIS Element software. The different surfaces for positively stained signals were quantified in 4 to 8 randomly chosen regions of interest (ROI of 0.3 mm²). In each ROI, the positive area was measured using pixel threshold on 8-bit converted images (ImageJ v1.43, Bethesda, Md., USA) and expressed as the area fraction of positive signal per ROI. Values were then averaged across all ROI and sections, and expressed as the average positive area per animal. The thickness of scar was measured on the ischemic boundary zone within the ipsilateral hemisphere on three sections stained for GFAP. The NF200 infiltration within the ischemic core represents the average of the length of axonal sprouts penetrating in the infarct area. The Dcx migration was measured on the ipsilateral hemisphere and represents the length of migration of Dcx positive neuroblasts along the Corpus Callosum.

Assessment of Infarct, Hemispheres and Cortex Volume

Quantification of infarct, ipsilateral and contralateral hemispheres and cortex was performed using a upright Leica DMLB microscope, equipped with hardware and software from Microbrightfield (Williston, Vt., USA). For each animal, every 10th coronal sections were stained for NeuN and Dapi and digitized using a computer-assisted analysis system, Stereo Investigator (Microbrightfield). The volumes were calculated by integrating the appropriate areas with the section interval thickness (250 μm). All measurements were averaged to obtain a single value per animal for every region of interest.

Evaluation of Blood-Brain Barrier (BBB) Permeability

BBB permeability was evaluated by assessing the extravasation of intravenously injected Evans blue dye in mouse brain. Briefly, the animals were anesthetized as previously described before injection of 2% Evans Blue dye/PBS (Sigma-Aldrich, St Louis, Mo.) into the left jugular vein (4 ml/kg). Brains were rapidly removed and each hemisphere placed separately in 1 ml of formamide and left to soak for 48 h at room temperature. The amount of extracted Evans Blue from the tissue was quantified by spectrophotometry. The absorbance of the supernatant solution was measured at 625 nm and a ratio ipsilateral/contralateral was obtained. Results were expressed as the relative absorbance (unit/g dry weight) and as a percentage of the PBS group.

Cytokine Analysis

Animals were stroked and injected with gel+nH five days later. The contralateral brain at 10-days (no stroke) and stroke only were used as positive and negative controls, respectively. At three and ten days after gel transplantation, animals were anesthetized and their blood flushed out with 20 mL cold PBS via transcardial perfusion. Brains were harvested and the infarct core with the injected gel were dissected and homogenized. A total of 1.5 mg/mL of sample was then diluted 1:1 with PBS+0.5% fetal bovine serum and quantified for their levels of cytokines using a multiplex Elisa analysis through the Bio-Plex kit (Bio-rad Laboratories Inc.) for the following cytokines: TNF-α, IL-6, IFN-γ, IL-1 α, IL-1 β, IL-2, IL-4 and IL-10.

Statistics

Tests were analyzed blindly to experimental condition. Animals were randomly assigned to control and treatment groups. Power analysis tool (Statistical Solutions LLC, Cottage Grove, Wis.) was employed to calculate sample size with the expected variance and changes in predicted proliferation and differentiation rates based on preliminary data. In each figure, data represent the average ±SEM (n=6-8) and P values were determined by One-way Anova, Tukey's post-hoc test. A value of P<0.05 was considered significant (Prism 5.03, graph Pad, San Diego, USA). For non-parametric variables (behavioral scores), multiple comparisons were performed using Kruskal-Wallis test with post-hoc Dunn test (n=12). In all the figures, *, **, ***, and **** indicate P<0.05, P<0.01, P<0.001, and P<0.0001 respectively, and represent statistical differences with all the other groups

While embodiments of the present invention have been shown and described, various modifications may be made without departing from the scope of the present invention. The invention, therefore, should not be limited, except to the following claims, and their equivalents.

1-20. (canceled)

21. A therapeutic hydrogel material for the treatment of stroke comprising a crosslinked hydrogel matrix consisting essentially of naked heparin nanoparticles distributed and entrained within the matrix and optional cell adhesion peptides covalently linked to the matrix, wherein the naked heparin nanoparticles within the crosslinked matrix: (1) do not exhibit blood thinning; (2) retain the ability to bind growth factors and/or cytokines; and (3) reduce localized inflammation.

22. The therapeutic hydrogel material of claim 21, wherein the crosslinked matrix is biodegradable.

23-25. (canceled)

26. The therapeutic hydrogel material of claim 21, wherein the naked heparin nanoparticles have a size range between about 90 nm to less than 1 μm.

27. The therapeutic hydrogel material of claim 21, wherein the hydrogel matrix comprises a biodegradable crosslinker.

28. The therapeutic hydrogel material of claim 27, wherein the biodegradable crosslinker comprises a matrix metalloproteinase (MMP) labile peptide.

29. The therapeutic hydrogel material of claim 21, wherein the hydrogel matrix comprises a cell adhesion peptide.

30. The therapeutic hydrogel material of claim 21, wherein the hydrogel matrix comprises hyaluronic acid functionalized with acrylamide groups.

31. The therapeutic hydrogel material of claim 21, wherein the hydrogel matrix is crosslinked in situ within a stroke cavity.

32. The therapeutic hydrogel material of claim 21, wherein the crosslinked matrix is formed by the co-delivery of uncrosslinked hydrogel and a crosslinking agent.

33. The therapeutic hydrogel material of claim 21, wherein the crosslinked matrix is formed by a mixture of uncrosslinked hydrogel and a crosslinking agent.

34. The therapeutic hydrogel material of claim 21, wherein the hydrogel comprises one of: a hyaluronic acid-based hydrogel, a poly(ethylene glycol)-based hydrogel, a poly(2-hydroxyethyl methacrylate) (PolyHEMA)-based hydrogel, an alginate-based hydrogel, a chitosan-based hydrogel, and a dextran-based hydrogel.

35. A method of treating brain tissue using the therapeutic hydrogel material of claim 21 comprising:
forming an artificial hole or opening in the skull of the mammal; and
delivering uncrosslinked hydrogel and a crosslinking agent to the brain tissue, wherein the uncrosslinked hydrogel transforms into the crosslinked matrix.

36. The method of claim 35, wherein the brain tissue comprises a stroke cavity.

37. The method of claim 35, wherein the uncrosslinked hydrogel and a crosslinking agent are co-delivered to the brain tissue.

38. The method of claim 35, wherein the uncrosslinked hydrogel and a crosslinking agent are mixed prior to delivery to the brain tissue.
